(19) United States
(12) Patent Application Publication
(10) Pub. No.: US 2011/0313158 A1
(43) Pub. Date: Dec. 22, 2011

(54) Two Component Recyclable Heterogeneous Catalyst, Process for Preparation Thereof and its Use for Preparation of Amines

(76) Inventors: Pravin R. Likhar, Hyderabad (IN); R. Arundhati, Hyderabad (IN); M. Lakshmi Kantam, Hyderabad (IN)

(21) Appl. No.: 13/203,228

(22) PCT Filed: Feb. 26, 2010
(86) PCT No.: PCT/IN2010/000116

§ 371 Date: Aug. 24, 2011

(30) Foreign Application Priority Data
Feb. 27, 2009 (IN) 386/DEL/2009

(51) Int. Cl. C07D 295/06; C07D 211/08; C07D 211/18; C07C 215/74; C07D 207/04; C07C 253/00; C07C 229/52; C07C 209/68; B01J 31/28; C07D 207/30

(52) U.S. Cl. 544/162; 502/172; 546/192; 546/232; 548/563; 548/577; 558/308; 562/457; 564/305; 564/411; 564/443

(57) ABSTRACT

The invention describes the development of highly efficient, recyclable two component system, CuAl-hydrotalcite/rac 1,1′-Binaphthalene-2,2′-diol catalytic system for the N-alkylation of electron deficient aryl chlorides in presence of potassium carbonate as a base at room temperature in 3-6 h, wherein the process is provided for the preparation of various secondary amines via C—N coupling reaction of aliphatic amines(aliphatic open chain, acyclic, benzyl amines and heterocyclic amines) with various aryl chlorides.

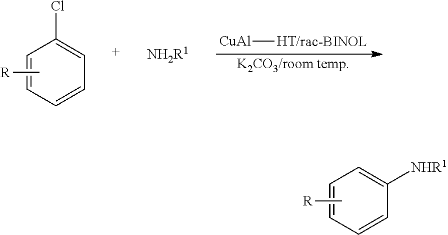

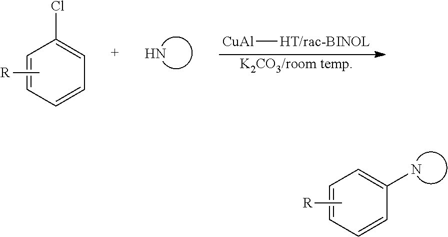

### FIELD OF THE INVENTION

The present invention relates to an improved process for the preparation of amines.

The present invention particularly relates to process for preparation of amines from aliphatic amines and aryl chlorides at room temperature. The present invention more particularly relates to the development of efficient and recyclable two component system composing copper-aluminum hydrotalcite/rac-1,1′-Binaphthalene-2,2′-diol for the N-alkylation of electron deficient aryl chlorides using K₂CO₃ as a base at room temperature. The amines used for the N-alkylation are aliphatic open chain amines (C5-C12), benzylamine, acyclic amines (C5-C7) and heterocyclic amines (pyrrole, pyrrolidine, piperidine, and morpholine).

### BACKGROUND OF THE INVENTION

Arylamines are widely used as versatile intermediates in polymers, pharmaceuticals and photographic materials. A large number of substituted N-arylamines are, used clinically as antihistamines, antihypertensive and anti-inflammatory drugs. They are also an important class of compounds in neuropharmaceuticals.

The exploration of new methodology for the synthesis of C—N bond via modern cross coupling chemistry was built at the beginning of the 20^{th} century with pioneering work of Ullmann and Irma Goldberg. Classic Ullmann and Goldberg protocol typically require harsh conditions such as high temperature, extended reaction time and in some cases stoichiometric amount of copper. To circumvent these problems, chemists have preferred the more recently developed palladium-catalyzed C—N bond forming reaction as a means to generate diverse array of arylated amines. However the palladium-catalyzed N-arylation also encounter some limitations. Furthermore copper is a cheaper metal and its environment friendly nature led chemists to use it at industrial scale.

The employment of chealating ligands has provided the major driving force behind the evolution of Cu-catalyzed C—N bond forming process. The first report concerning the intentional use of exogenous ligand is focused on 1,10 phenanthroline. The discovery and development of the catalytic path of N-arylation by Buchwald and Taillefer with bromo- and iodoarenes using copper in presence of basic ligand generated greater interest in industry. Among the haloarenes, the N-arylation of chloroarenes is of importance, since this reaction involving C—Cl activation contributes to the fundamental understanding of the reactivity of such very stable bond. Most importantly, they are cheaper and widely available than their bromide or iodide counterparts.

Numerous procedures for the synthesis of the secondary amines are described using transition metal catalyzed cross coupling reaction between aryl halides and aliphatic amines. Among these, most of the cross coupling reactions are dominated by palladium catalysts. The conventional Ullmann reaction is poorly suited to the arylation of aliphatic amines and therefore various ligand-assisted methods immediately brought forward for this purpose. In the past few years, the copper-catalyzed Ullmann reactions have shown renaissance because of the correct choice of copper sources and appropriate ligands. Now this two component catalyst system is popular and is used to develop highly efficient ligand supported copper catalyzed arylation reactions. In this direction, great progress has been made in the N-arylation reaction of amines/amides, still a simple and general procedure for the copper catalyzed coupling of aliphatic amines with aryl halides under mild reaction condition has remained elusive/unexplored.

Copper catalyzed N-arylation of aliphatic amines may be achieved by various methods, one possible way is N-alkylation of aryl bromide with primary alkyl amines using Copper-diethylsalicylamide in presence of K₃PO₄ as a base at 90° C. for 18-22 h. The reaction protocol for this method is described by Buchwald et al. in Org. Lett. 2003, 5, 793.

Ma et al. [J. Org. Chem. 2005, 70, 5164] were able to demonstrate the use of CuI-amino acid catalyzed coupling reaction of aryl iodide with aliphatic primary amines at 40-110° C. using K₂CO₃ as a base.

Wan et al. [Tetrahedron 2005, 61, 903] were able to show the use of copper bromide and phosphoramidite in the N-arylation of alkylamines and heterocyclic amines with aryl iodide using Cs₂CO₃ at 90° C. for 24 h.

Despite the synthetic elegance and high turnover number, these coupling reactions suffer from serious limitations of using the expensive bromo- and iodoarenes that precluded the wide use in industry. By employing the recyclable heterogeneous catalytic system, chloroarenes as starting materials and low reaction temperature (reaction performed at room temperature), and this process will certainly be favored as an economic and industrially feasible process due to easy recovery of the catalyst and low cost of chloroarenes when compared with bromo- and iodoarenes.

It was therefore desirable to provide a process for the preparation of N-arylated compounds that start with inexpensive aryl chlorides and catalyzed by recyclable copper source and commercially available ligand at room temperature.

Buchwald et al. [J. Am. Chem. Soc. 2006, 128, 8742] and Fu et al. [J. Org. Chem. 2007, 72, 672] were independently able to show the N-arylation of aliphatic amines at room temperature catalyzed by CuI/β-diketone as catalyst and Cs₂CO₃ as base and CuBr/rac 1,1′-Binaphthalene-2,2′-diol as catalyst and K₃PO₄ as a base respectively. In both of these reports, reactions were performed under homogeneous conditions and aryl iodides and aryl bromides were used for the N-arylation of aliphatic amines.

Recently, we have shown the activation of C—Cl bond of aryl chlorides for the synthesis of amines using various amines in the presence of K₂CO₃ as base at relatively high temperature (Tetrahedron Letter 2007, 48, 3911).

### OBJECTIVES OF THE INVENTION

The principal objective of the present invention is to provide an alternative and efficient process for the synthesis of secondary amines by coupling aliphatic amines with aryl chlorides in presence of base and recyclable two components catalytic system composed of hydrotalcite-like compounds and rac-1,1′-Binaphthalene-2,2′-diol as a supporting ligand at room temperature.

Another object of the present invention is the coupling reaction of aliphatic amines (aliphatic open chain C4-C13; acyclic amines C5-C8; benzyl amines; heterocyclic amines) with aryl chlorides.

Still another object of the present invention is the usage of non-corrosive and low cost heterogenous catalyst, hydrotalcite-like compounds as catalysts.

Still another object of the present invention is to use copper aluminum hydrotalcite (Cu—Al—HT) with a Cu/Al atomic ratio of 2.0:1, 2.5:1 and 3:1 (CuAl 3.0-HT; CuAl 2.5-HT; CuAl 2.0-HT).

Still another object of the present invention is to use hydrotalcite catalyst dried at 65° C./12 h.

Still another object of the present invention is the use of anhydrous base with particle size ranges from 20-50μ and vacuum dried for 8 h at 100° C.

Still another object of the present invention is to use of racemic 1,1′-Binaphthalene-2,2′-diol as a supporting ligand with Cu—Al—HT in the ratio of 1:20 to 1:30.

Still another object of the present invention is the reaction performed at room temperature (25-35° C.) in 2-5 h.

Still yet another object of the present invention is to use of the reusable catalyst.

### SUMMARY OF INVENTION

Accordingly the present invention provides a two component recyclable heterogeneous catalyst comprising; CuAl—HT/rac 1,1′-Binaphthalene-2,2′-diol wherein the ratio of Cu:Al varies from 2:1 to 3:1 and the molar ratio of CuAl—HT to rac 1,1′-Binaphthalene-2,2′-diol varies from 20:1 to 30:1.

In an embodiment of the present invention wherein the catalyst may be useful for preparation of amines.

In an embodiment of the present invention wherein the catalyst is recyclable and reusable for at least next five consecutive cycles without loss of catalytic activity.

Accordingly the present invention also provides a process for preparation of catalyst as claimed in claim 1 wherein the process comprising: adding rac 1,1′-Binaphthalene-2,2′-diol to the suspension of CuAl—HT in a solvent selected from a group consisting of alkyl (C1-C2) and aromatic (C6) nitriles, under stirring and continued the stirring for a period ranging between 30 to 50 hr at a temperature ranging between 25-35° C. under inert atmosphere, filtering the suspension followed by washing with water and corresponding alkyl/aromatic nitrile, drying the catalyst under vacuo.

Accordingly the present invention also provides an improved process for preparation of amines using the catalyst as claimed in claim 1 wherein the process steps comprises; reacting an amine with aryl chlorides in presence of highly efficient and recyclable two component, CuAl—HT/rac 1,1′-Binaphthalene-2,2′-diol catalyst and a base selected from the group consisting of alkali metal hydroxide/carbonate/alkoxide at a temperature ranging between 25-35° C. for a period ranging between of 3-6 h under vigorous stirring condition, separating the product from the crude mixture using the known methods.

In another embodiment of the present invention wherein the amines used may be selected from a group consisting of aliphatic open chain, acyclic amines, benzyl amines and heterocyclic amines.

In still another embodiment of the present invention wherein the aryl chlorides used may be selected from an electron deficient and functional group substituted at ortho, meta and para position.

In a further embodiment of the present invention wherein the ratio of Cu to Al is 2.5:1 in the catalyst used for the preparation of amine.

In yet another embodiment of the present invention wherein, the two component catalyst system prepared by using CuAl—HT/rac 1,1′-Binaphthalene-2,2′-diol in the molar ratio (27:1).

In an embodiment of the present invention wherein the catalyst concentration is 15 mol % based on Cu metal with respect to aryl chloride.

In an embodiment of the present invention, wherein the base concentration is 2 equivalent with respect to aryl chloride.

The novelty of present invention lies in the use of cheap heterogeneous catalyst composed of CuAl—HT and rac 1,1′-Binaphthalene-2,2′-diol for the first time for the N-alkylation of aryl chlorides. The present invention provides a process for the synthesis of various secondary amines via coupling reactions of aliphatic amines (aliphatic open chain C4-C12; alicyclic amines C5-C7; benzyl amities; heterocyclic amines) with electron deficient aryl chlorides (electronically activated) in the presence of a base wherein the base is alkali metal hydroxide/carbonate/alkoxide. The solid base catalyst of general formula, [M(II)₁₋ₓ M(III)ₓ (OH)₂]ⁿ⁻ Aⁿ⁻_{x/n·y}H₂O, where M(II) and M(III) are divalent and trivalent cations such as Cu²⁺, Mg²⁺ and Al³⁺ respectively, Aⁿ⁻ the interlayer anion such as Cl⁻, NO₃⁻, CO₃²⁻ etc., and x=0.1-0.33.

The goal of the present invention is to provide a simple method in which, in particular, coupling of aliphatic amines with aryl chlorides to afford high selectivity and high yield of secondary amines, using a cheap heterogeneous CuAl—HT/rac 1,1′-Binaphthalene-2,2′-diol catalyst in a single step at room temperature.

### DETAILED DESCRIPTION OF THE INVENTION

The invention describes the development of highly efficient, recyclable two component system, CuAl-hydrotalcite/rac 1,1′-Binaphthalene-2,2′-diol catalytic system for the N-alkylation of electron deficient aryl chlorides in presence of potassium carbonate as a base at room temperature in 3-6 h, wherein the process is provided for the preparation of various secondary amines via C—N coupling reaction of aliphatic amines(aliphatic open chain, acyclic, benzyl amines and heterocyclic amities) with various aryl chlorides.

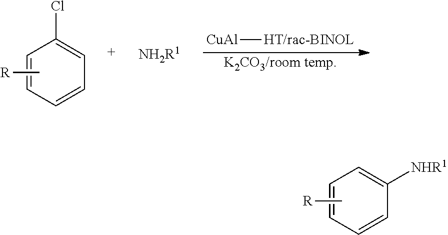

R=4-NO₂; 3-NO₂; 2-NO₂; 4-CN; 3-CN; 2-CN; 4-Cl; 3-Cl; 4-COOH; 4-OH
R¹=aliphatic open chain C5-C12; alicyclic C5-C7 ring; benzylamine

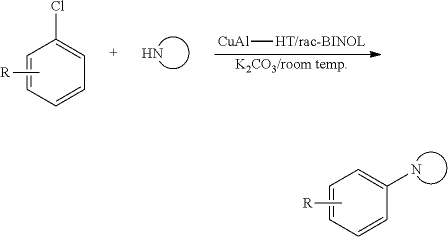

R=4-NO₂; 3-NO₂; 2-NO₂; 4-CN; 3-CN; 2-CN; 4-Cl; 3-Cl; 4-COON; 4-OH
Heterocyclic amines=1H-pyrrole, pyrrolidine, piperidine, morpholine

The present invention describes a batch process, which comprises a development of highly efficient, recoverable and recyclable two component catalyst system composed of Cu—Al HT/rac 1,1′-Binaphthalene-2,2′-diol for N-alkylation of aryl chlorides with aliphatic amines in presence of base at room temperature. In this invention aliphatic amines used are aliphatic open chain amines, C4-C13; alicyclic amines, C5-C8; benzylamine and heterocyclic amines and aryl chlorides used are mainly electronically activated (ortho, meta and para substituted functional group). The base is selected from mainly carbonate, hydroxide and alkoxide of alkali metals, and heterogeneous catalyst, CuAl-hydrotalcite where the ratio of Cu:Al varies from 2:1 to 3:1 and the ratio of CuAl—HT to rac 1,1′-Binaphthalene-2,2′-diol varies from 20:1 to 30:1. The reaction is carried out in the presence of 16.377 mol % based on Cu metal with respect to aryl chlorides at room temperature (25-35° C.) for 2-6 h under continuous stirring using amines as self-solvent under air atmosphere. The process of the invention overcomes the disadvantage of the prior art enumerated above since the work up is simple, and the catalyst is recoverable and recyclable with consistent activity for several cycles. The use of cheap inorganic base, inexpensive catalyst and different amines for C—N bond formation with aryl chlorides provides secondary amines as products in good to excellent yield in a single step.

Generally the ratio of Cu to Al in the catalyst is 2.5:1 and the quantity used in the reactions is 16.377 mol % based on Cu metal with respect to aryl chlorides. The active catalyst composed of CuAl—HT/rac 1,1′-Binaphthalene-2,2′-diol and the molar ratio of CuAl—HT to rac 1,1′-Binaphthalene-2,2′-diol is 27:1. The catalyst used in the reactions can be recovered by simple filtration and reused for number of cycles with consistent activity.

The reaction is preferably carried out in the presence of separately prepared two component catalyst system (CuAl—HT/rac1,1′-Binaphthalene-2,2′-diol), base at room temperature. The process comprises the unique activation of C—Cl bond of aryl chlorides to facilitate simultaneous C—N bond formation with aliphatic amines in presence of base in a single pot.

Incidentally this forms the first report on the N-alkylation of aryl chlorides using aliphatic amines in high yields in single pot using highly efficient, recoverable and recyclable cheap heterogeneous catalyst at room temperature. The consistent activity obtained for several cycle makes the process economical and possible for commercial realization.

The compounds prepared by using this method are N-benzyl-4-nitrobenzenamine, N-benzyl-2-nitrobenzenamine, 4-(benzylamino)benzonitrile, 2-(benzylamino)benzonitrile, 3-(benzylamino)benzonitrile, 4-(benzylamino)benzaldehyde, 2-(benzylamino)benzaldehyde, 5-(benzylamino)benzene-1,3-dialdehyde, 4-(benzylamino)benzoic acid, 4-(benzylamino)phenol, 2-(benzylamino)phenol, 4-nitro-N-pentylbenzenamine, N-hexyl-4-nitrobenzene amine, 4-nitro-N-octylbenzenamine, N-dodecyl-4-nitrobenzenamine, 2-nitro-N-pentylbenzenamine, N-hexyl-2-nitrobenzenamine, 2-nitro-N-octylbenzenamine, N-dodecyl-2-nitrobenzenamine, 4-(octylamino)benzonitrile, 2-(octylamino)benzoic acid, 2-(octylamino)phenol, 4-chloro-N-octylbenzenamine, 3-chloro-N-octylbenzenamine, 3-(octylamino)benzonitrile, 3-nitro-N-octylbenzeneamine, N-(4-nitrophenyl)cycloheptanmine, N-cyclohexyl-4-nitrobenzenamine, N-cyclohexyl-2-nitrobenzenamine, 4-(cyclohexylamino)benzonitrile, 2-(cyclohexylamino)benzonitrile, 4-(cyclohexylamino)benzoic acid, 4-(cyclohexylamino)phenol, 4-chloro-N-cyclohexylbenzenamine, N-cyclohexyl-3-nitro benzenamine, 3-(cyclohexylamino)benzonitrile, 3-chloro-N-cyclohexylbenzenamine, N-cyclopentyl-4-nitrobenzenamine, N-cyclohexyl-4-nitrobenzenamine, N-cyclopentyl-2-nitrobenzenamine, 4-(cyanopentylamino)benzonitrile, 2-(cyanopentylamino) benzonitrile, 4-(cyanopentylamino)benzoic acid, 1-phenylpyrrolidine, 1-(4-nitro phenyl)pyrrolidine, 1(4-chlorophenyl)pyrrolidine, 1-phenylpiperidine, 4-(4-nitro phenyl)morpholine, 4(2-nitrophenyl)morpholine, 1-(4-nitrophenyl)-1H-pyrrole, 1-(4-nitrophenyl)piperidine, 1(2-nitrophenyl)piperidine, 1(2-nitrophenyl)-1H-pyrrole, 4-(1H-pyrrol-1-yl)benzonitrile, 2(1H-pyrrol-1-yl)benzonitrile, 3-(1H-pyrrol-1-yl)benzonitrile, 4-(1H-pyrrol-1-yl)benzald-ehyde, 3-nitro-4-(1H-pyrrol-1-yl)benzaldehyde, 4-(1H-pyrrol-1-yl)benzoic acid.

In an embodiment of the present invention, the heterogeneous catalyst used is CuAl-hydrotalcite/rac 1,1′-Binaphthalene-2,2′-diol.

In an embodiment of the present invention, N-alkylation is performed using aliphatic amines (aliphatic open chain amines, C4-C13; acyclic amines, C5-C8; benzyl amines, and heterocyclic amines) with easily accessible and cheap aryl chlorides.

In an embodiment of the present invention, anhydrous powdered K₂CO₃ base is added at the start of the reaction.

In an embodiment of the present invention, the two component catalyst system composed of CuAl—HT (2.5)/rac 1,1′-Binaphthalene-2,2′-diol in the molar ratio of 27:1

In an embodiment of the present invention, the mole ratio of aliphatic amines to aryl chlorides used as 1.5:1.0 without using any additional solvents.

In an embodiment of the present invention, the reaction is effected at room temperature (˜30° C.).

In still another embodiment of the present invention, the amount of base is 2 mole per mole of aryl chlorides.

In still another embodiment of the present invention wherein the catalyst is inert, eco-friendly and non-toxic.

In still another embodiment of the present invention wherein the catalyst is immiscible and stable in organic as well as in aqueous phase.

In still another embodiment of the present invention wherein there is absolutely no leaching of the metal content during the reaction as well as during the workup.

### Scientific Explanation

The process of the invention comprises the activation of C—Cl bond to facilitate simultaneous attack by nucleophile to obtain secondary amine in excellent yield in a single pot. The employment of chealating ligands has provided the major driving force behind the evolution of Cu-catalyzed C—N bond forming process. The spectroscopic data revealed that the rac 1,1′-Binaphthalene-2,2′-diol interacts with the copper present on the surface of CuAl—HT through coordination of oxygen and thereby making an active catalyst for the C—N bond formation reactions. Copper present in the interlayer of CuAl—HT is not used in the coordination with rac 1,1′-Binaphthalene-2,2′-diol and thus maintain low stoichiometry with copper. The hard donor oxygen of the rac 1,1′-Binaphthalene-2,2′-diol ligand forms strong coordination bond with metal center. The catalytic cycle of coupling reactions of amines and aryl chlorides may involve first generation of amine based anion in presence of base and basic catalyst at room temperature through base abstraction of the proton from the amine producing anion, act as a nucleophile. Thus the nucleophilic attack by anion at aromatic carbocation further proceeded to coupling reaction to produce alkyl-arylamine.

The catalyst can be recovered by simple filtration and can be reused for the next cycle. Therefore the invented strategy offers an environmentally acceptable and extremely convenient heterogeneous catalytic process for the synthesis of secondary amines from the coupling reaction of various aliphatic amines and aryl chlorides in batch processes.

The following examples are given by way of illustration of the present invention and therefore should not be construed to limit the scope of the present invention.

### Examples 1

### Catalyst Preparation

### A: Cu—Al Hydrotalcite (2.5:1)

Cu:Al hydrotalcite (2.5:1) is prepared as follows: About 200 ml of deionised water was taken into a 1 lit. four necked round bottom flask and stirred at 25° C. with a overhead mechanical stirrer. A mixture of solution of Cu(NO₃)₂.3H₂O (90.57 g, 0.375 moles) and Al(NO₃)₃.9H₂O (56.27 g, 0.0.15 moles) in deionised water (140 mL) and the aqueous solutions of NaOH (42.97 g, 2.09 moles) and Na₂CO₃ (33.12 g, 0.312 moles) in deionised water were added simultaneously into the round bottom flask. The pH of the reaction mixture was maintained constantly (7-8) by continuous addition of the base solution. The resulting slurry was aged at 70° C. for two hours. The solid product was isolated by filtration, washed thoroughly with deionised water (to make base free) and dried under 70° C. for 15 h.

B: Cu—Al Hydrotalcite/rac-1,1′-Binaphthalene-2,2′-diol,

A solution of rac 1,1′-Binaphthalene-2,2′-diol 0.5 g (0.2 mmol) in 5 mL of CH₃CN, was added drop wise to the suspension of CuAl—HT, (1.0 g) in CH₃CN (25 mL), under stirring condition. The mixture was stirred under inert atmosphere for 48 h at 25° C. The solid suspension was filtered, washed with deionized water followed by CH₃CN and dried in vacuo overnight at 25° C., yielding the CuAl—HT/rac 1,1′-Binaphthalene-2,2′-diol two component system as a blue powder. The ratio of CuAl—HT to rac 1,1′-Binaphthalene-2,2′-diol was observed as 27:1.

General procedure of N-Alkylation of aryl chlorides using aliphatic open chain amines and benzylamines

In a 50 mL round bottom flask, aryl chloride (6.35 mmol, 1.0 equiv), linear amine (9.5 mmol, 1.5 equiv), CuAl—HT/rac 1,1′-Binaphthalene-2,2′-diol (0.25 g, 1.04 mmol of Cu metal; 16.377 mol % w. r. t. aryl chloride), K₂CO₃ (12.7 mmol, 2.0 equiv) were taken and the mixture was stirred at room temperature (25-35° C.) for an appropriate time under aerobic condition. The progress of the reaction was monitored by TLC and on completion of the reaction, the reaction mixture was centrifuged to separate the catalyst, the solid residue was washed several times with ethyl acetate to make the catalyst free from organic matter, the centrifugate was then washed with water and dried over anhyd. Na₂SO₄, the reaction mixture was concentrated under reduced pressure to give the crude product. The crude product was purified by column chromatography on silica (60-120 mesh) gel using ethyl acetate:hexane (10:90) as an eluent to afford the corresponding product secondary amine. Some of the few examples are illustrated given below:

### Example 2

Following general procedure for N-alkylation of aryl chloride using aliphatic open chain amines and benzylamines, aryl chloride used as 4-nitrochlorobenzene and amine used as benzylamine. The yield of isolated product, N-benzyl-4-nitrobenzenamine is found to be 89% in 4 h.

### Example 3

Following general procedure for N-alkylation of aryl chloride using aliphatic open chain amines and benzylamines, aryl chloride used as 2-nitrochlorobenzene and amine used as benzylamine. The yield of isolated product, N-benzyl-2-nitrobenzenamine is found to be 99% in 1.5 h.

### Example 4

Following general procedure for N-alkylation of aryl chloride using aliphatic open chain amines and benzylamines, aryl chloride used as 4-cyanochlorobenzene and amine used as benzylamine. The yield of isolated product, 4-(benzylamino)benzonitrile is found to be 65% in 5 h.

### Example 5

Following general procedure for N-alkylation of aryl chloride using aliphatic open chain amines and benzylamines, aryl chloride used as 2-cyanochlorobenzene and amine used as benzylamine. The yield of isolated product, 2-(benzylamino)benzonitrile is found to be 70% in 3 h.

### Example 6

Following general procedure for N-alkylation of aryl chloride using aliphatic open chain amines and benzylamines, aryl chloride used as 3-cyanochlorobenzene and amine used as benzylamine. The yield of isolated product, 3-(benzylamino)benzonitrile is found to be 90% in 2 h.

### Example 7

Following general procedure for N-alkylation of aryl chloride using aliphatic open chain amines and benzylamines, aryl chloride used as 4-chlorobenzaldehyde and amine used as benzylamine. The yield of isolated product, 4-(benzylamino)benzaldehyde is found to be 85% in 1.5 h.

### Example 8

Following general procedure for N-alkylation of aryl chloride using aliphatic open chain amines and benzylamines, aryl chloride used as 2-chlorobenzaldehyde and amine used as benzylamine. The yield of isolated product, 2-(benzylamino)benzaldehyde is found to be 95% in 1 h.

### Example 9

Following general procedure for N-alkylation of aryl chloride using aliphatic open chain amines and benzylamines, aryl chloride used as 3-formyl-5-chlorobenzaldehyde and amine used as benzylamine. The yield of isolated product, 5-(benzylamino)benzene-1,3-dialdehyde is found to be 99% in 1.5 h.

### Example 10

Following general procedure for N-alkylation of aryl chloride using aliphatic open chain amines and benzylamines, aryl chloride used as 4-chlorobenzoic acid and amine used as benzylamine. The yield of isolated product, 4-(benzylamino)benzoic acid is found to be 99% in 1 h.

### Example 11

Following general procedure for N-alkylation of aryl chloride using aliphatic open chain amines and benzylamines, aryl chloride used as 4-chlorophenol and amine used as benzylamine. The yield of isolated product, 4-(benzylamino)phenol is found to be 63% in 4 h.

### Example 12

Following general procedure for N-alkylation of aryl chloride using aliphatic open chain amines and benzylamines, aryl chloride used as 2-chlorophenol and amine used as benzylamine. The yield of isolated product, 2-(benzylamino)phenol is found to be 75% in 2 h.

### Example 13

Following general procedure for N-alkylation of aryl chloride using aliphatic open chain amines and benzylamines, aryl chloride used as 4-nitrochlorobenzene and amine used as pentylamine. The yield of isolated product, 4-nitro-N-pentylbenzenamine is found to be 99% in 4 h.

### Example 14

Following general procedure for N-alkylation of aryl chloride using aliphatic open chain amines and benzylamines, aryl chloride used as 4-nitrochlorobenzene and amine used as hexylamine. The yield of isolated product, N-hexyl-4-nitrobenzenamine is found to be 98% in 1.5 h.

### Example 15

Following general procedure for N-alkylation of aryl chloride using aliphatic open chain amines and benzylamines, aryl chloride used as 4-nitrochlorobenzene and amine used as octylamine. The yield of isolated product, 4-nitro-N-octylbenzenamine is found to be 95% in 3 h.

### Example 16

Following general procedure for N-alkylation of aryl chloride using aliphatic open chain amities and benzylamines, aryl chloride used as 4-nitrochlorobenzene and amine used as dodecylamine. The yield of isolated product, N-dodecyl-4-nitrobenzenamine. The isolated yield is found to be 99% in 2 h.

### Example 17

Following general procedure for N-alkylation of aryl chloride using aliphatic open chain amines and benzylamines, aryl chloride used as 2-nitrochlorobenzene and amine used as pentylamine. The yield of isolated product, 2-nitro-N-pentylbenzenamine is found to be 97% in 1 h.

### Example 18

Following general procedure for N-alkylation of aryl chloride using aliphatic open chain amines and benzylamines, aryl chloride used as 2-nitrochlorobenzene and amine used as hexylamine. The yield of isolated product, N-hexyl-2-nitrobenzenamine is found to be 99% in 1 h.

### Example 19

Following general procedure for N-alkylation of aryl chloride using aliphatic open chain amines and benzylamines aryl chloride used as 2-nitrochlorobenzene and amine used as octylamine. The yield of isolated product, 2-nitro-N-octylbenzenamine is found to be 99% in 1 h.

### Example 20

Following general procedure for N-alkylation of aryl chloride using aliphatic open chain amines and benzylamines, aryl chloride used as 2-nitrochlorobenzene and amine used as dodecylamine. The yield of isolated product, N-dodecyl-2-nitrobenzenamine is found to be 99% in 1 h.

### Example 21

Following general procedure for N-alkylation of aryl chloride using aliphatic open chain amines and benzylamines, aryl chloride used as 4-cyanochlorobenzene and amine used as octylamine. The yield of isolated product, 4-(octylamino)benzonitrile is found to be 82% in 3 h.

### Example 22

Following general procedure for N-alkylation of aryl chloride using aliphatic open chain amines and benzylamines, aryl chloride used as 2-chlorobenzoic acid and amine used as octylamine. The yield of isolated product, 2-(octylamino)benzoic acid is found to be 93% in 1 h.

### Example 23

Following general procedure for N-alkylation of aryl chloride using aliphatic open chain amines and benzylamines, aryl chloride used as 2-chlorophenol and amine used as octylamine. The yield of isolated product, 2-(octylamino)phenol is found to be 65% in 4 h.

### Example 24

Following general procedure for N-alkylation of aryl chloride using aliphatic open chain amines and benzylamines, aryl chloride used as 1,4-dichlorobenzene and amine used as octylamine. The yield of isolated product, 4-chloro-N-octylbenzenamine is found to be 72% in 5 h.

### Example 25

Following general procedure for N-alkylation of aryl chloride using aliphatic open chain amines and benzylamines, aryl chloride used as 1,3-dichlorobenzene and amine used as octylamine. The yield of isolated product, 3-chloro-N-octylbenzenamine is found to be 82% in 2 h.

### Example 26

Following general procedure for N-alkylation of aryl chloride using aliphatic open chain amines and benzylamines, aryl chloride used as 3-cyanochlorobenzene and amine used as octylamine. The yield of isolated product, 3-(octylamino)benzonitrile is found to be 75% in 3 h.

### Example 27

Following general procedure for N-alkylation of aryl chloride using aliphatic open chain amines and benzylamines, aryl chloride used as 3-nitrochlorobenzene and amine used as octylamine. The yield of isolated product, 3-nitro-N-octylbenzeneamine is found to be 93% in 1 h.

### General Procedure for N-Alkylation of Aryl Chlorides Using Alicyclic Amines:

In a 50 mL round bottom flask, aryl chloride (6.35 mmol, 1.0 equiv), alicyclic amine (9.5 mmol, 1.5 equiv), CuAl—HT/rac 1,1′-Binaphthalene-2,2′-diol (0.25 g, 1.04 mmol of Cu metal; 16.377 mol % w. r. t. aryl chloride) as prepared in Example 1, K₂CO₃ (12.7 mmol, 2.0 equiv) were taken and the mixture was stirred at room temperature (25-35° C.) for an appropriate time under aerobic condition. The progress of the reaction was monitored by TLC and on completion of the reaction, the reaction mixture was centrifuged to separate the catalyst, the solid residue was washed several times with ethyl acetate to make the catalyst free from organic matter, the centrifugate was then washed with water and dried over anhyd. Na₂SO₄, the reaction mixture was concentrated under reduced pressure to give the crude product. The crude product was purified by column chromatography on silica (60-120 mesh) gel using ethyl acetate:hexane (10:90) as an eluent to afford the corresponding product secondary amine. Some of the few examples are illustrated given below:

### Example 28

Following general procedure for N-alkylation of aryl chloride using alicyclic amines, aryl chloride used as 4-nitrochlorobenezene and amine used as cycloheptylamine. The yield of isolated product, N-(4-nitrophenyl)cycloheptanmine is found to be 80% in 5 h.

### Example 29

Following general procedure for N-alkylation of aryl chloride using alicyclic amines, aryl chloride used as 4-nitrochlorobenezene and amine used as cyclohexylamine. The yield of isolated product, N-cyclohexyl-4-nitrobenzenamine is found to be 95% in 5 h.

### Example 30

Following general procedure for N-alkylation of aryl chloride using alicyclic amines, aryl chloride used as 2-nitrochlorobenezene and amine used as cyclohexylamine. The yield of isolated product, N-cyclohexyl-2-nitrobenzenamine is found to be 99% in 3 h.

### Example 31

Following general procedure for N-alkylation of aryl chloride using alicyclic amines, aryl chloride used as 4-cyanochlorobenezene and amine used as cyclohexylamine. The yield of isolated product, 4-(cyclohexylamino)benzonitrile is found to be 69% in 6 h.

### Example 32

Following general procedure for N-alkylation of aryl chloride using alicyclic amines, aryl chloride used as 2-cyanochlorobenezene and amine used as cyclohexylamine. The yield of isolated product, 2-(cyclohexylamino)benzonitrile is found to be 81% in 4 h.

### Example 33

Following general procedure for N-alkylation of aryl chloride using alicyclic amines, aryl chloride used as 4-chlorobenezoic acid and amine used as cyclohexylamine. The yield of isolated product, 4-(cyclohexylamino)benzoic acid is found to be 90% in 5 h.

### Example 34

Following general procedure for N-alkylation of aryl chloride using alicyclic amines, aryl chloride used as 4-chlorophenol and amine used as cyclohexylamine. The yield of isolated product, 4-(cyclohexylamino)phenol is found to be 85% in 6 h.

### Example 35

Following general procedure for N-alkylation of aryl chloride using alicyclic amines, aryl chloride used as 1,4-dichlorobenezene and amine used as cyclohexylamine. The yield of isolated product, 4-chloro-N-cyclohexylbenzenamine is found to be 95% in 5 h.

### Example 36

Following general procedure for N-alkylation of aryl chloride using alicyclic amines, aryl chloride used as 3-Nitrochlorobenezene and amine used as cyclohexylamine. The yield of isolated product, N-cyclohexyl-3-nitrobenzenamine is found to be 90% in 3 h.

### Example 37

Following general procedure for N-alkylation of aryl chloride using alicyclic amines, aryl chloride used as 3-cyanochlorobenezene and amine used as cyclohexylamine. The yield of isolated product, 3-(cyclohexylamino)benzonitrile is found to be 81% in 3 h.

### Example 38

Following general procedure for N-alkylation of aryl chloride as stated in Example 28, aryl chloride used as 1,3-dichlorobenezene and amine used as cyclohexylamine. The yield of isolated product, 3-chloro-N-cyclohexylbenzenamine is found to be 84% in 4 h.

### Example 39

Following general procedure for N-alkylation of aryl chloride using alicyclic amines, aryl chloride used as 4-nitrochlorobenezene and amine used as cyclopentylamine. The yield of isolated product, N-cyclopentyl-4-nitrobenzenamine is found to be 95% in 5 h.

### Example 40

Following general procedure for N-alkylation of aryl chloride using alicyclic amines, aryl chloride used as 4-nitrochlorobenezene and amine used as cyclohexylamine. The yield of isolated product, N-cyclohexyl-4-nitrobenzenamine is found to be 85% in 4 h.

### Example 41

Following general procedure for N-alkylation of aryl chloride using alicyclic amines, aryl chloride used as 2-nitrochlorobenezene and amine used as cyclopentylamine. The yield of isolated product, N-cyclopentyl-2-nitrobenzenamine is found to be 91% in 3 h.

### Example 42

Following general procedure for N-alkylation of aryl chloride using alicyclic amines, aryl chloride used as 4-cyanochlorobenezene and amine used as cyclopentylamine. The yield of isolated product, 4-(cyclopentylamino)benzonitrile is found to be 95% in 5 h. 4-(cyclopentylamino)benzonitrile

### Example 43

Following general procedure for N-alkylation of aryl chloride using alicyclic amines, aryl chloride used as 2-cyanochlorobenezene and amine used as cyclopentylamine. The yield of isolated product, 2-(cyclopentylamino)benzonitrile is found to be 65% in 3 h.

### Example 44

Following general procedure for N-alkylation of aryl chloride using alicyclic amines, aryl chloride used as 4-chlorobenezoic acid and amine used as cyclopentylamine. The yield of isolated product, 4-(cyclopentylamino)benzoic acid is found to be 75% in 4 h.

### General Procedure for N-Alkylation of Aryl Chlorides Using Heterocyclic Amines:

In a 50 mL round bottom flask, aryl chloride (6.35 mmol, 1.0 equiv), heterocyclic amine (9.5 mmol, 1.5 equiv), CuAl—HT/rac 1 μl′-Binaphthalene-2,2′-diol (0.25 g, 1.04 mmol of Cu metal; 16.377 mol % w. r. t. aryl chloride) as prepared in Example 1, K₂CO₃ (12.7 mmol, 2.0 equiv) were taken and the mixture was stirred at room temperature (25-35° C.) for an appropriate time under aerobic condition. The progress of the reaction was monitored by TLC and on completion of the reaction, the reaction mixture was centrifuged to separate the catalyst, the solid residue was washed several times with ethyl acetate to make the catalyst free from organic matter, the centrifugate was then washed with water and dried over anhyd. Na₂SO₄, the reaction mixture was concentrated under reduced pressure to give the crude product. The crude product was purified by column chromatography on silica (60-120 mesh) gel using ethyl acetate:hexane (10:90) as an eluent to afford the corresponding product secondary amine. Some of the few examples are illustrated given below:

### Example 45

Following general procedure for N-alkylation of aryl chloride using heterocyclic amines: used as chlorobenezene and amine used as pyrrolidine. The yield of isolated product, 1-phenylpyrrolidine is found to be 67% in 9 h.

### Example 46

Following general procedure for N-alkylation of aryl chloride using heterocyclic amines, aryl chloride used as 4-nitrochlorobenezene and amine used as pyrrolidine. The yield of isolated product, 1-(4-nitrophenyl)pyrrolidine is found to be 71% in 4 h.

### Example 47

Following general procedure for N-alkylation of aryl chloride using heterocyclic amines, aryl chloride used as 1,4-dichlorobenezene and amine used as pyrrolidine. The yield of isolated product, 1-(4-chlorophenyl)pyrrolidine is found to be 82% in 2 h.

### Example 48

Following general procedure for N-alkylation of aryl chloride using heterocyclic amines, aryl chloride used as chlorobenezene and amine used as piperidine. The yield of isolated product, 1-phenylpiperidine is found to be 67% in 6 h.

### Example 49

Following general procedure for N-alkylation of aryl chloride using heterocyclic amines, aryl chloride used as 4-nitrochlorobenezene and amine used as morpholine. The yield of isolated product, 4-(4-nitrophenyl)morpholine is found to be 78% in 8 h.

### Example 50

Following general procedure for N-alkylation of aryl chloride using heterocyclic amines, aryl chloride used as 2-nitrochlorobenezene and amine used as morpholine. The yield of isolated product, 4-(2-nitrophenyl)morpholine is found to be 86% in 8 h.

### Example 51

Following general procedure for N-alkylation of aryl chloride using heterocyclic amines, aryl chloride used as 4-nitrochlorobenezene and amine used as 1H-pyrrole. The yield of isolated product, 1-(4-nitrophenyl)-1H-pyrrole is found to be 43% in 6 h.

### Example 52

Following general procedure for N-alkylation of aryl chloride using heterocyclic amines, aryl chloride used as 4-nitrochlorobenezene and amine used as piperidine. The yield of isolated product, 1-(4-nitrophenyl)piperidine is found to be 66% in 7 h.

### Example 53

Following general procedure for N-alkylation of aryl chloride using heterocyclic amines, aryl chloride used as 2-nitrochlorobenezene and amine used as piperidine. The yield of isolated product, 1-(2-nitrophenyl)piperidine is found to be 99% in 2 h.

### Example 54

Following general procedure for N-alkylation of aryl chloride using heterocyclic amines, aryl chloride used as 2-nitrochlorobenezene and amine used as 1H-pyrrole. The yield of isolated product, 1-(2-nitrophenyl)-1H-pyrrole is found to be 99% in 3 h.

### Example 55

Following general procedure for N-alkylation of aryl, chloride using heterocyclic amines, aryl chloride used as 4-cyanochlorobenezene and amine used as 1H-pyrrole. The yield of isolated product, 4-(1H-pyrrol-1-yl)benzonitrile is found to be 55% in 7 h.

### Example 56

Following general procedure for N-alkylation of aryl chloride as stated in Example 46, aryl chloride used as 2-cyanochlorobenezene and amine used as 1H-pyrrole. The yield of isolated product, 2-(1H-pyrrol-1-yl)benzonitrile is found to be 81% in 5 h.

### Example 57

Following general procedure for N-alkylation of aryl chloride using heterocyclic amines, aryl chloride used as 3-cyanochlorobenezene and amine used as 1H-pyrrole. The yield of isolated product, 3-(1H-pyrrol-1-yl)benzonitrile is found to be 85% in 4 h.

### Example 58

Following general procedure for N-alkylation of aryl chloride using heterocyclic amines, aryl chloride used as 4-chlorobenezaldehyde and amine used as 1H-pyrrole. The yield of isolated product, 4-(1H-pyrrol-1-yl)benzaldehyde is found to be 82% in 3 h.

### Example 59

Following general procedure for N-alkylation of aryl chloride using heterocyclic amines, aryl chloride used as 2-nitrochlorobenezaldehyde and amine used as 1H-pyrrole. The yield of isolated product, 2-nitro-3-(1H-pyrrol-1-yl)benzaldehyde is found to be 89% in 2 h.

### Example 60

Following general procedure for N-alkylation of aryl chloride using heterocyclic amines, aryl chloride used as 1-chlorobenzoic acid and amine used as 1H-pyrrole. The yield of isolated product, 4-(1H-pyrrol-1-yl)benzoic acid is found to be 60% in 2 h.

As mentioned earlier, we have recently shown Cu—Al—HT catalyzed activation of C—Cl bond of aryl chlorides for the synthesis of amines using various amines in the presence of, K2CO3 as base at relatively high temperature in absence of rac 1,1′-Binaphthalene-2,2′-diol (Tetrahedron Letter 2007, 48, 3911). The following are some of the examples which show the effect of rac 1,1′-Binaphthalene-2,2′-diol on N-alkylation reactions. For eg. by using the rac 1,1′-Binaphthalene-2,2′-diol in the C—N couplings of 4-nitrochlorobenzene; 2-nitrochlorobenzene; 4-cyanochlorobenzene; 4-formylchlorobenzene; 4-chlorobenzoic acid, not only C—Cl bonds activate at room temperature but the yields of corresponding products also increase. Similarly, several aryl halides were capable to couple with new amines (linear aliphatic amines, alicyclic amines and heterocyclic amines) and afforded good to excellent yields of corresponding product using these reaction parameters or else no reactions were observed in absence of rac1,1′-Binaphthalene-2,2′-diol.

The main advantages of the present invention are:
1. The present invention comprises highly selective, efficient and recyclable two component catalyst system composed of CuAl—HT/rac 1,1′-Binaphthalene-2,2′-diol for the preparation of secondary amines via C—N bond formation reaction of aliphatic amines with aryl chloride in presence of base at room temperature (25-35° C.) in 2-5 h. The product yield obtained in most of the cases are in the range of 90-99% in isolated form without any operational difficulty experienced during the course of the reaction.
2. The present process envisages the use of cheap and easily accessible aryl chlorides as arylating agents for N— alkylation of various amines at room temperature for the first time.
3. CuAl—HT/rac1,1′-Binaphthalene-2,2′-diol, two component catalyst system used for N-alkylation of aryl chlorides is recyclable and reusable for at least next five consecutive cycles without loss of catalytic activity.
4. The present process envisages optimal use of CuAl—HT/rac1,1′-Binaphthalene-2,2′-diol, two component catalyst system and base to ensure highest conversion and selectivity.
5. An eco-friendly and very simple synthetic protocol is developed using cheap and non-corrosive CuAl—HT/rac1,1′-Binaphthalene-2,2′-diol, two component catalyst system.
6. The reaction conditions are extremely mild (reactions performed at room temperature, under air and in very short time).
7. The selectivity, yield and purity of N-arylated products in this process are quite high.
8. Monitoring of the reaction and subsequent work-up procedures are easy.
9. The overall process is economical.
10. The catalyst used is inert, eco-friendly and non-toxic.
11. The catalyst used is immiscible and stable in organic as well as in aqueous phase.
12. There is absolutely no leaching of the metal content during the reaction as well as during the workup.

We claim:

1. A two component recyclable heterogeneous catalyst comprising; CuAl—HT and rac 1,1′-Binaphthalene-2,2′-diol wherein the ratio of Cu:Al varies from 2:1 to 3:1 and the molar ratio of CuAl—HT to rac 1,1′-Binaphthalene-2,2′-diol varies from 20:1 to 30:1.

2. A catalyst as claimed in claim 1 wherein the catalyst is useful for preparation of amines.

3. A catalyst as claimed in claim 1 wherein the catalyst is recyclable and reusable for at least next five consecutive cycles without loss of catalytic activity.

4. A process for preparation of catalyst as claimed in claim 1 wherein the process comprising: adding rac 1,1′-Binaphthalene-2,2′-diol to the suspension of CuAl—HT in a solvent selected from a group consisting of alkyl (C1-C2) and aromatic (C6) nitriles, under stirring for 30 to 50 hr at a temperature ranging between 25-35° C. under inert atmosphere, filtering the suspension followed by washing with water and corresponding alkyl/aromatic nitrile, drying the catalyst under vacuo.

5. An process for preparation of amines using the catalyst as claimed in claim 1 wherein the process steps comprises; reacting an amine with aryl chlorides in presence of highly efficient and recyclable two component, CuAl—HT and rac 1,1′-Binaphthalene-2,2′-diol catalyst and a base selected from alkali metal hydroxide/carbonate/alkoxide at a temperature ranging between 25-35° C. for a period ranging between of 3-6 h under vigorous stirring condition, separating the product from the crude mixture using the known methods.

6. A process as claimed in claim 5, wherein the amines used is selected from a group consisting of aliphatic open chain, acyclic amines, benzyl amines and heterocyclic amines.

7. A process as claimed in claim 5, wherein the aryl chlorides used is selected from an electron deficient and functional group substituted at ortho, meta and para position.

8. A process as claimed in claim 5, wherein the ratio of Cu to Al is 2.5:1 in the catalyst used for the preparation of amine.

9. A process as claimed in claim 5 wherein, the two component catalyst system prepared by using CuAl—HT and rac 1,1′-Binaphthalene-2,2′-diol in the molar ratio 27:1.

10. A process as claimed in claim 5, wherein the catalyst concentration is 16.377 mol % based on Cu metal with respect to aryl chloride.

11. A process as claimed in claim 5, wherein the base concentration is 2 equivalent with respect to aryl chloride.

12. A process as claimed in claim 5, wherein the representative compounds prepared by the process comprising:
1. N-benzyl-4-nitrobenzenamine
2. 2, N-benzyl-2-nitrobenzenamine
3. 4-(benzylamino)benzonitrile
4. 2-(benzylamino)benzonitrile
5. 3-(benzylamino)benzonitrile
6. 4-(benzylamino)benzaldehyde
7. 2-(benzylamino)benzaldehyde
8. 5-(benzylamino)benzene-1,3-dialdehyde
9. 4-(benzylamino)benzoic acid
10. 4-(benzylamino)phenol
11. 2-(benzylamino)phenol
12. 4-nitro-N-pentylbenzenamine
13. N-hexyl-4-nitrobenzenamine
14. 4-nitro-N-octylbenzenamine
15. N-dodecyl-4-nitrobenzenamine.
16. 2-nitro-N-pentylbenzenamine
17. N-hexyl-2-nitrobenzenamine
18. 2-nitro-N-octylbenzenamine
19. N-dodecyl-2-nitrobenzenamine
20. 4-(octylamino)benzonitrile
21. 2-(octylamino)benzoic acid
22. 2-(octylamino)phenol
23. 4-chloro-N-octylbenzenamine
24. 3-chloro-N-octylbenzenamine
25. 3-(octylamino)benzonitrile
26. 3-nitro-N-octylbenzeneamine
27. N-(4-nitrophenyl)cycloheptanmine
28. N-cyclohexyl-4-nitrobenzenamine
29. N-cyclohexyl-2-nitrobenzenamine
30. 4-(cyclohexylamino)benzonitrile
31. 2-(cyclohexylamino)benzonitrile
32. 4-(cyclohexylamino)benzoic acid
33. 4-(cyclohexylamino)phenol
34. 4-chloro-N-cyclohexylbenzenamine
35. N-cyclohexyl-3-nitrobenzenamine
36. 3-(cyclohexylamino)benzonitrile
37. 3-chloro-N-cyclohexylbenzenamine
38. N-cyclopentyl-4-nitrobenzenamine
39. N-cyclohexyl-4-nitrobenzenamine
40. N-cyclopentyl-2-nitrobenzenamine
41. 4-(cyclopentylamino)benzonitrile
42. 2-(cyclopentylamino)benzonitrile
43. 4-(cyclopentylamino)benzoic acid
44. 1-phenylpyrrolidine
45. 1-(4-nitrophenyl)pyrrolidine
46. 1-(4-chlorophenyl)pyrrolidine
47. 1-phenylpiperidine
48. 4-(4-nitrophenyl)morpholine
49. 4-(2-nitrophenyl)morpholine
50. 1-(4-nitrophenyl)-1H-pyrrole
51. 1-(4-nitrophenyl)piperidine
52. 1-(2-nitrophenyl)piperidine
53. 1-(2-nitrophenyl)-1H-pyrrole
54. 4-(1H-pyrrol-1-yl)benzonitrile
55. 2-(1H-pyrrol-1-yl)benzonitrile
56. 3-(1H-pyrrol-1-yl)benzonitrile
57. 4-(1H-pyrrol-1-yl)benzaldehyde
58. 2-nitro-3-(1H-pyrrol-1-yl)benzaldehyde
59. 4-(1H-pyrrol-1-yl)benzoic acid
